SEQUENCE LISTING (14 sequences)

SEQ ID NO: 1 - protein, 9 aa, Artificial sequence
  CDR1
GHFSNLAVN

SEQ ID NO: 2 - protein, 17 aa, Artificial sequence
  CDR2
GILWSGGSTFYADSVKG

SEQ ID NO: 3 - protein, 3 aa, Artificial sequence
  CDR3
GTN

SEQ ID NO: 4 - protein, 10 aa, Artificial sequence
  HCDR1
GYTFTNYGMN

SEQ ID NO: 5 - protein, 17 aa, Artificial sequence
  HCDR2
WINTYTGEPTYAADFKR

SEQ ID NO: 6 - protein, 14 aa, Artificial sequence
  HCDR3
YPHYYGSSHWYFDV

SEQ ID NO: 7 - protein, 11 aa, Artificial sequence
  LCDR1
SASQDISNYLN

SEQ ID NO: 8 - protein, 7 aa, Artificial sequence
  LCDR2
FTSSLHS

SEQ ID NO: 9 - protein, 9 aa, Artificial sequence
  LCDR3
QQYSTVPWT

SEQ ID NO: 10 - protein, 111 aa, Artificial sequence
  VHH
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSS

SEQ ID NO: 11 - protein, 123 aa, Artificial sequence [sequence omitted]

SEQ ID NO: 12 - protein, 107 aa, Artificial sequence [sequence omitted]

SEQ ID NO: 13 - protein, 452 aa, Artificial sequence
  HC
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 335 aa, Artificial sequence
  LC
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0242645 A1
(43) Pub. Date: Aug. 3, 2023

(54) A BISPECIFIC ANTI-PD-L1/VEGF ANTIBODY AND USES THEREOF

(71) Applicant: WUXI BIOLOGICS (SHANGHAI) CO. LTD., Shanghai (CN)

(72) Inventors: Zhuozhi WANG, Shanghai (CN); Yunying CHEN, Shanghai (CN); Dong LI, Shanghai (CN); Jing LI, Shanghai (CN)

(21) Appl. No.: 17/792,386

(22) PCT Filed: Jan. 19, 2021
(86) PCT No.: PCT/CN2021/072584

§ 371 Date: Jul. 12, 2022

(30) Foreign Application Priority Data
Jan. 21, 2020 (WO) PCT/CN2020/073497

(51) Int. Cl. C07K 16/28; C07K 16/22; A61P 35/00; A61K 45/06; A61K 39/395

(52) U.S. Cl. CPC C07K 16/2827; C07K 16/22; A61P 35/00; A61K 45/06; A61K 39/3955; A61K 2039/505

(57) ABSTRACT

Provided are bispecific anti-VEGF×PD-L1 antibody or antigen-binding portion thereof, methods of producing the bispecific antibody or antigen-binding portion thereof, and methods of treating diseases or conditions using the bispecific antibody or antigen-binding portion thereof.

### CROSS-REFERENCE TO RELATED APPLICATION

The application claims priority to International application PCT/CN2020/073497, filed on Jan. 21, 2020, the entire contents of which are hereby incorporated by reference.

### SEQUENCE LISTING

The present application is filed with a Sequence Listing in electronic form. The entire contents of the Sequence Listing are hereby incorporated by reference.

### FIELD

The present disclosure generally relates to bispecific anti-PD-L1×VEGF antibodies, a method for preparing the same and uses thereof.

### BACKGROUND

Angiogenesis is essential for the growth of tumor and the development of metastasis. Controlling tumor-associated angiogenesis is a promising strategy for cancer therapy. Vascular endothelial growth factor (VEGF) is the key mediator of angiogenesis, and has been validated in various types of human cancers. Tumor cells release growth factors such as VEGF that bind to nearby endothelial cells, which initiates a signaling cascade that stimulates endothelial cells to divide and form new blood vessels. VEGF signaling through its receptors VEGFR plays a critical role in angiogenesis and growth of many solid tumors. Antiangiogenic drugs, such as Avastin (Bevacizumab) which targets the VEGF pathway, have achieved a success in clinic.

On the other hand, targeting immune checkpoint molecules, such as programmed death ligand 1 (PD-L1) or its receptor, programmed death 1 (PD-1), has shown promising clinical success. PD-L1 expression strongly correlates with unfavorable prognosis in various types of cancers. Anti-PD-L1 antibody can target PD-L1 expressed on tumor cells and tumor-infiltrating immune cells, and prevent binding to PD-1 and B7.1 on the surface of T cells, and also enable the activation of T cells as well as recruit other T cells to attack the tumor, then empower the immune system to fight multiple types of cancer.

Anti-VEGF therapy, in addition to its established anti-angiogenic effects, may further enhance anti-PD-1/PD-L1 therapy's ability to restore anti-cancer immunity, by inhibiting VEGF-related immunosuppression, promoting T-cell tumor infiltration and enabling priming and activation of T-cell responses against tumor antigens. Therefore, developing VEGF and PD-L1 bispecific antibodies which combine anti-angiogenesis therapy and immune checkpoint inhibition together could achieve promising results in cancer therapy.

Despite the obvious benefit of targeting VEGF and also targeting PD-1/PD-L1 therapies, there is still a significant unmet need. 15%-20% patients do not respond to anti-VEGF treatment, and increasing evidence has indicated that prolonged use of anti-VEGF agents for cancer therapy promotes tumor resistance. 3%-9% patients develop immunogenicity of the treatment. And also there exists the limited overall survival time extension and limited safety issue, including changes in bone morphology, glomerulopathy with inflammation in kidney, and decreased vacuolation with inflammation in adrenal gland. Immune checkpoint inhibitors blocking the PD-1/PD-L1 pathway, such as nivolumab, pembrolizumab and atezolizumab, represent a standard treatment option for patients with multiple cancers. However, with a response rate of 14-23% in unselected populations and 16-48% in patients with PD-L1-expressing tumors, these drugs offer improved outcomes in some patients, but not all.

Therefore, there is great need to develop novel anti-PD-L1/anti-VEGF bispecific antibodies. In the present disclosure, a bispecific antibody that could simultaneously bind to human PD-L1 and VEGF with high affinity, block both PD-1/PD-L1 and VEGF/VEGFR signaling, and display superior anti-tumor efficacy has been generated.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

In one aspect, the present disclosure provides a bispecific antibody or antigen-binding portion thereof, comprising a PD-L1 antigen-binding moiety associated with a VEGF antigen-binding moiety, wherein:

the PD-L1 antigen-binding moiety comprises: a complementarity determining region (CDR) 1 comprising SEQ ID NO: 1, a CDR2 comprising SEQ ID NO: 2, and a CDR3 comprising SEQ ID NO: 3, and

the VEGF antigen-binding moiety comprises: a heavy chain complementarity determining region (HCDR) 1 comprising SEQ ID NO: 4, a HCDR2 comprising SEQ ID NO: 5, a HCDR3 comprising SEQ ID NO: 6, a light chain complementarity determining region (LCDR) 1 comprising SEQ ID NO: 7, a LCDR2 comprising SEQ ID NO: 8, and a LCDR3 comprising SEQ ID NO: 9.

In certain embodiments, the PD-L1 antigen-binding moiety as disclosed herein comprises: a variable domain comprising the amino acid sequence of SEQ ID NO: 10 or an amino acid sequence at least 85%, 90%, or 95% identical to SEQ ID NO: 10.

In certain embodiments, the VEGF antigen-binding moiety as disclosed herein comprises:

a heavy chain variable domain comprising the amino acid sequence of SEQ ID NO: 11 or an amino acid sequence at least 85%, 90%, or 95% identical to SEQ ID NO: 11; and

a light chain variable domain comprising the amino acid sequence of SEQ ID NO:12 or an amino acid sequence at least 85%, 90%, or 95% identical to SEQ ID NO: 12.

In certain embodiments, the PD-L1 antigen-binding moiety is fused to the N terminal of the VEGF antigen-binding moiety. In some other embodiments, the PD-L1 antigen-binding moiety is fused to the C terminal of the VEGF antigen-binding moiety.

In certain embodiments, the PD-L1 antigen-binding moiety is from a single-domain antibody (sdAb), such as a VHH antibody. The VHH may be derived from a camelid animal, comprising an alpaca or a llama. Preferably, the VHH is a humanized VHH.

In certain embodiments, the PD-L1 antigen-binding moiety is operably linked to the N terminal of the light chain or heavy chain of the VEGF antigen-binding moiety, optionally via a linker. The linker may comprise or consist of 1 to 4 copies of GGGGS (G4S), for example, the linker may be (G4S)2.

In certain embodiments, the bispecific antibody or antigen-binding portion thereof as disclosed herein comprises a heavy chain and a light chain, wherein:

the heavy chain comprises domains operably linked as in VH-CH1-hinge-Fc, wherein the VH-CH1 is from the VEGF antigen binding moiety; and

the light chain comprises domains operably linked as in VHH-VL-CL, wherein the VHH is from the PD-L1 antigen binding moiety and the VL-CL is from the VEGF antigen binding moiety.

In certain embodiments, the Fc region is a human IgG Fc region, preferably a human IgG1 Fc region.

In certain embodiments, the bispecific antibody or antigen-binding portion thereof as disclosed herein comprise a heavy chain comprising SEQ ID NO: 13 and a light chain comprising SEQ ID NO: 14.

In certain embodiments, the bispecific antibody or antigen-binding portion thereof as disclosed herein is a humanized antibody.

In one aspect, the present disclosure provides an isolated nucleic acid molecule, comprising a nucleic acid sequence encoding the bispecific antibody or the antigen-binding portion thereof as disclosed herein.

In one aspect, the present disclosure provides a vector comprising the nucleic acid molecule as disclosed herein. In one aspect, the present disclosure provides a host cell comprising the nucleic acid molecule or the vector as disclosed herein.

In one aspect, the present disclosure provides a pharmaceutical composition comprising the bispecific antibody or the antigen-binding portion thereof as disclosed herein and a pharmaceutically acceptable carrier.

In one aspect, the present disclosure provides a method for producing the bispecific antibody or the antigen-binding portion thereof as disclosed herein, comprising the steps of:
expressing the antibody or the antigen-binding portion thereof in the host cell; and
isolating the antibody or antigen-binding portion thereof from the host cell.

In one aspect, the present disclosure provides a method for modulating an immune response in a subject, comprising administering to the subject the bispecific antibody or the antigen-binding portion thereof or the pharmaceutical composition as disclosed herein to the subject.

In one aspect, the present disclosure provides a method for inhibiting growth of tumor cells in a subject, comprising administering an effective amount of the bispecific antibody or the antigen-binding portion thereof or the pharmaceutical composition as disclosed herein to the subject.

In one aspect, the present disclosure provides a method for preventing or treating cancer in a subject, comprising administering an effective amount of the bispecific antibody or the antigen-binding portion thereof or the pharmaceutical composition to the subject. The cancer may be selected from colon cancer, colorectal cancer, breast cancer, lung cancer, cervical cancer, renal cancer, glioblastoma, ovarian cancer, pancreatic cancer, prostate cancer, esophageal cancer, gastric cancer, lymphoma, melanoma, liver cancer, and head and neck cancer. In certain embodiments, the cancer is colon cancer or colorectal cancer.

In certain embodiments, the bispecific antibody or antigen-binding portion thereof as disclosed herein may be administered in combination with a chemotherapeutic agent, radiation and/or other agents for use in cancer immunotherapy.

In one aspect, the present disclosure provides a bispecific antibody or antigen-binding portion thereof as disclosed herein for use

i) in the modulation of PD-L1/VEGF related immune responses;

ii) in enhancing T cell proliferation and cytokine production; and/or

iii) in stimulating an immune response or function, such as boosting the immune response against cancer cells.

In one aspect, the present disclosure provides the bispecific antibody or antigen-binding portion thereof as disclosed herein for use in diagnosing, preventing or treating cancers.

In one aspect, the present disclosure provides use of the bispecific antibody or antigen-binding portion thereof as disclosed herein in the manufacture of a medicament for modulating an immune response or inhibiting growth of tumor cells in a subject.

In one aspect, the present disclosure provides use of the bispecific antibody or antigen-binding portion thereof as disclosed herein in the manufacture of a medicament for treating or preventing cancers.

In one aspect, the present disclosure provides a kit comprising the bispecific antibody or antigen-binding portion thereof as disclosed herein. The kit can be used for detection, diagnosis, prognosis, or treatment of a disease or condition, such as cancer.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Further, the contents of all references, patents and published patent applications cited throughout this application are incorporated herein in entirety by reference.

### BRIEF DESCRIPTION OF FIGURES

FIG. 1 shows a schematic representation of the format of W3256 antibody.

FIG. 2 shows SDS-PAGE of W3256 antibody (lane 1). NR: Non-reducing status, R: reducing status in NuPAGE (Novex 4-12% Bis-Tris) gel. M, PageRuler™ Unstained Protein Ladder.

FIG. 3 shows the result of HPLC-SEC of W3256 antibody.

FIG. 4 shows the DSF profile of W3256 antibody.

FIG. 5 shows the ELISA binding result of W3256 antibody to human VEGF (same as cyno VEGF). WBP332-1.80.12×Ab.hIgG1 is an isotype control.

FIG. 6 shows the FACS binding result of W3256 antibody to human PD-L1.

FIG. 7 shows the dual binding result of W3256 antibody to VEGF and then PD-L1.

FIG. 8 shows the dual binding of W3256 antibody to PD-L1 and then VEGF.

FIG. 9 shows the FACS binding result of W3256 antibody to cynomolgus PD-L1.

FIG. 10 shows the ELISA binding result of W3256 antibody to mouse VEGF.

FIG. 11 shows the FACS binding result of W3256 antibody to mouse PD-L1.

FIGS. 12-13 show the SPR sensorgram of W3256 antibody binding to human PD-L1 (FIG. 12) and VEGF (FIG. 13).

FIGS. 14-15 show the competition of W3256 antibody to human VEGFR1 on human VEGF binding (FIG. 14), and the competition of W3256 antibody to human PD-1 on human PD-L1 binding (FIG. 15).

FIGS. 16-17 show the competition of W3256 antibody to mouse VEGFR1 on human VEGF binding (FIG. 16), and the competition of W3256 antibody to mouse PD-1 on mouse PD-L1 binding (FIG. 17).

FIG. 18 shows the inhibition of antibodies on HUVEC cells proliferation.

FIG. 19 shows the effect of antibodies on reporter gene assay.

FIG. 20 shows the effect of antibodies on hCD4+ T cell IFN-γ secretion in MLR. “Combo” refers to the combination of WBP325-BMK3.uIgG1 and W315-BMK8.uIgG1K(RKNA).

FIG. 21 shows mouse PK analysis of W3256 antibody in total IgG binding assay after a single intravenous injection of equal molar dose of antibody. “mpk” refers to the abbreviation of “mg/kg”.

FIG. 22 shows mouse PK analysis of W3256 antibody in dual-antigen binding assay after a single intravenous injection of equal molar dose of antibody.

FIG. 23 shows the efficacy of antibodies in PBMC-RKO cancer model in mice.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “antibody” or “Ab,” herein is used in the broadest sense, which encompasses various antibody structures, including polyclonal antibodies, monospecific and multispecific antibodies (e.g. bispecific antibodies). A native intact antibody generally is a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (VH) and a heavy chain constant region (CH). A heavy chain constant region consists of 3 domains (CH1, CH2 and CH3). Each light chain consists of a light chain variable region (VL) and a light chain constant region (CL). VH and VL region can further be divided into hypervariable regions (called complementary determining regions (CDR)), which are interspaced by relatively conservative regions (called framework region (FR)). Each VH and VL consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Chothia et al., (1989) Nature 342:878-883. Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. In certain embodiments, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

The term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

The term “antigen-binding moiety” as used herein refers to an antibody fragment formed from a portion of an antibody comprising one or more CDRs, or any other antibody fragment that binds to an antigen but does not comprise an intact native antibody structure. Different from the term “antigen binding site” which generally refers to the variable domains, an antigen-binding moiety may comprise constant domains in addition to variable domains. Examples of antigen-binding moiety include, without limitation, a variable domain, a variable region, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may be a Fab fragment or a VHH antibody. In some embodiments, an antigen-binding moiety may comprise one or more CDRs from a particular human antibody grafted to a framework region from one or more different human antibodies. For more and detailed formats of antigen-binding moiety are described in Spiess et al., Molecular Immunology, 67(2), pp. 95-106 (2015), and Brinkman et al., mAbs, 9(2), pp. 182-212 (2017), which are incorporated herein by their entirety.

“Fab” with regard to an antibody refers to that portion of the antibody consisting of a single light chain (both variable and constant regions) associating to the variable region and first constant region of a single heavy chain by a disulfide bond.

“Fc” with regard to an antibody refers to that portion of the antibody comprising the second (CH2) and third (CH3) constant regions of a first heavy chain bound to the second and third constant regions of a second heavy chain via disulfide bonding. When referring to a Fc region, depending on the context, it may refer to one chain or both chains of the Fc region. The Fc portion of the antibody is responsible for various effector functions such as ADCC, and CDC, but does not function in antigen binding. The capacity of antibodies to initiate and regulate effector functions through their Fc domain is a key component of their in vivo protective activity. Although the neutralizing activity of antibodies has been previously considered to be solely the outcome of Fab-antigen interactions, it has become apparent that their in vivo activity is highly dependent on interactions of the IgG Fc domain with its cognate receptors, Fcγ receptors (FcγRs), expressed on the surface of effector leukocytes.

The term “PD-L1”, also known as programmed death-ligand 1, is a 40 kDa type 1 transmembrane protein that has been speculated to play a major role in suppressing the adaptive arm of immune system. PD-L1 is the principal ligand of programmed death 1 (PD-1), a coinhibitory receptor that can be constitutively expressed or induced in myeloid, lymphoid, normal epithelial cells and in cancer. The term “PD-L1” as used herein, when referring to the amino acid sequence of PD-L1 protein, including full-length PD-L1 protein, or the extracellular domain of PD-L1 (PD-L1 ECD) or fragment containing PD-L1 ECD; Fusion protein of PD-L1 ECD, for example, fragment fused with IgG Fc from mice or human (mFc or hFc) is also included. Moreover, as understood by a person skilled in the art, PD-L1 protein would also include those into which mutations of amino acid sequence are naturally or artificially introduced (including but not limited to replacement, deletion and/or addition) without affecting the biological functions.

The term “an antibody that binds PD-L1” or an “anti-PD-L1 antibody” as used herein includes antibodies and antigen-binding fragments thereof that specifically recognize PD-L1. The antibodies and antigen-binding fragments of the present disclosure may bind soluble PD-L1 protein and/or cell surface expressed PD-L1. Soluble PD-L1 includes natural PD-L1 proteins as well as recombinant PD-L1 protein variants that lack a transmembrane domain or are otherwise unassociated with a cell membrane. As used herein, the expression “anti-PD-L1 antibody” includes both monovalent antibodies with a single specificity, as well as bispecific antibodies comprising a first antigen-binding site that binds PD-L1 and a second antigen-binding site that binds a second (target) antigen, wherein the anti-PD-L1 antigen-binding site comprises any of the HCVR/LCVR or CDR sequences as set forth in Table A herein. Examples of anti-PD-L1 bispecific antibodies are described elsewhere herein. The term “antigen-binding molecule” includes antibodies and antigen-binding fragments of antibodies, including, e.g., bispecific antibodies.

The term “VEGF” (vascular endothelial growth factor, also known as VEGF-A), is a signal protein produced by cells that stimulates the formation of blood vessels. VEGF is a sub-family of growth factors, the platelet-derived growth factor family of cysteine-knot growth factors. They are important signaling proteins involved in both vasculogenesis (the de novo formation of the embryonic circulatory system) and angiogenesis (the growth of blood vessels from pre-existing vasculature). The VEGF family includes VEGF-A, VEGF-B, VEGF-C, VEGF-D, P1GF (placental growth factor), VEGF-E (Orf-VEGF), and Trimeresurus flavoviridis svVEGF.

The term “VEGF receptor” or “VEGFR” as used herein refers to receptors for vascular endothelial growth factor (VEGF). There are three main subtypes of VEGFR, numbered 1, 2 and 3. The VEGF receptors may be membrane-bound or soluble, depending on alternative splicing. Among the VEGF receptors, VEGFR-1 binds VEGF-A, P1GF, and VEGF-B.

As used herein, a “bispecific antibody” refers to an artificial antibody, which has fragments derived from two different monoclonal antibodies and is capable of binding to two different epitopes. The two epitopes may present on the same antigen, or they may present on two different antigens.

The term “bispecific antigen-binding molecule” means a protein, polypeptide or molecular complex comprising at least a first antigen-binding domain (also referred to as a first antigen-binding site herein) and a second antigen-binding domain (also referred to as a second antigen-binding site herein). In some embodiments, the “bispecific antigen-binding molecule” is a “bispecific antibody”. Each antigen-binding domain within the bispecific antibody comprises at least one CDR that alone, or in combination with one or more additional CDRs and/or FRs, specifically binds to a particular antigen. In the context of the present disclosure, the first antigen-binding site specifically binds to a first antigen (e.g., PD-L1), and the second antigen-binding site specifically binds to a second, distinct antigen (e.g., VEGF).

The term “anti-PD-L1/anti-VEGF antibody”, “anti-PD-L1/anti-VEGF bispecific antibody”, “antibody against PD-L1 and VEGF”, “anti-PD-L1×VEGF bispecific antibody”, “PD-L1×VEGF antibody”, as used herein interchangeably, refers to a bispecific antibody that specifically binds to PD-L1 and VEGF.

The term “monoclonal antibody” or “mAb”, as used herein, refer to a preparation of antibody molecules of single molecular composition. A monoclonal antibody displays a single binding specificity and affinity for a particular epitope.

The term “chimeric antibody”, as used herein, refers to antibodies in which the variable region sequences are derived from one species and the constant region sequences are derived from another species, such as an antibody in which the variable region sequences are derived from a mouse antibody and the constant region sequences are derived from a human antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “IC₅₀”, as used herein, which is also termed as “half maximal inhibitory concentration” is a measure of the potency of a substance in inhibiting a specific biological or biochemical function. In the context of the application, IC₅₀ is expressed in the unit of “nM”.

The ability of “inhibit binding,” as used herein, refers to the ability of an antibody or antigen-binding fragment thereof to inhibit the binding of two molecules (eg, human PD-L1/VEGF and human PD-1/VEGFR) to any detectable level. In certain embodiments, the binding of the two molecules can be inhibited by the antibodies at an IC₅₀ of no more than 50 nM, no more than 30 nM, no more than 10 nM, no more than 5 nM, no more than 1 nM or even less.

The term “epitope,” as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. See, for example, Epitope Mapping Protocols in Methods in Molecular Biology, Vol. 66, G. E. Morris, Ed. (1996).

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds a PD-L1/VEGF protein is substantially free of antibodies that specifically bind antigens other than PD-L1/VEGF proteins). An isolated antibody that specifically binds a human PD-L1/VEGF protein may, however, have cross-reactivity to other antigens, such as PD-L1/VEGF proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human PD-L1/VEGF gene was transfected into 293F cells.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09.). Instruments for carrying out FACS are known to those of skill in the art and are commercially available to the public. Examples of such instruments include FACS Star Plus, FACScan and FAC Sort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For instance, the “an effective amount,” when used in connection with treatment of PD-L1/VEGF-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

Bispecific Antibodies and Antigen-Binding Portions Thereof

In certain embodiments, the antibodies and antigen-binding portions thereof provided herein are bispecific. In some embodiments, the bispecific antibodies and antigen-binding portions thereof provided herein have a first specificity for PD-L1, and a second specificity for VEGF.

According to certain exemplary embodiments, the present disclosure includes a bispecific antibody or the antigen-binding portion thereof, comprising a first antigen-binding moiety that specifically binds to PD-L1 and a second antigen-binding moiety that specifically binds to VEGF. Such antibodies may be referred to herein as, e.g., “anti-VEGF/anti-PD-L1” or “anti-PD-L1/VEGF,” or “anti-PD-L1×VEGF” or “PD-L1×VEGF” bispecific antibodies, or other similar terminology.

The bispecific antibodies of the disclosure could bind to human PD-L1 and human VEGF with high affinity. The binding of an antibody of the disclosure to PD-L1 or VEGF can be assessed using one or more techniques well established in the art, for instance, ELISA. The binding specificity of an antibody of the disclosure can also be determined by monitoring binding of the antibody to cells expressing a PD-L1 protein or a VEGF protein, e.g., flow cytometry. For example, an antibody can be tested by a flow cytometry assay in which the antibody is reacted with a cell line that expresses human PD-L1, such as CHO cells that have been transfected to express PD-L1 on their cell surface. Additionally or alternatively, the binding of the antibody, including the binding kinetics (e.g., K_{D} value) can be tested in BIAcore binding assays. Still other suitable binding assays include ELISA or FACS assays, for example using a recombinant PD-L1 protein.

For instance, an antibody of the disclosure binds to a human PD-L1 protein or human VEGF protein with a K_{D} of 1×10⁻⁷ M or less, a K_{D} of 5×10⁻⁸ M or less, a K_{D} of 2×10⁻⁸ M or less, a K_{D} of 1×10⁻⁸ M or less, a K_{D} of 5×10⁻⁹ M or less, a K_{D} of 4×10⁻⁹ M or less, a K_{D} of 3×10⁻⁹ M or less, a K_{D} of 2×10⁹ M or less, a K_{D} of 1×10⁻⁹ M or less, a K_{D} of 5×10⁻¹⁰ M or less, or a K_{D} of 1×10⁻¹⁰ M or less, as measured by Surface Plasmon Resonance.

As demonstrated in the Example section, the bispecific antibodies of the disclosure could bind to human PD-L1 and human VEGF (same as cyno VEGF) with a high affinity; bind to cyno and mouse PD-L1; effectively block both PD-1/PD-L1 and VEGFR/VEGF signaling pathways, e.g. with an IC50 of nM grade; block VEGF induced HUVEC proliferation; and produce strong agonistic effect on cytokine secretion.

The PD-L1 Antigen-Binding Moiety

The PD-L1 binding moiety as defined herein may have various formats (e.g. VHH, scFv, Fab), as long as it can specifically bind to the antigen. Generally, the PD-L1 binding moiety comprised in the bispecific antibody is derived from a monospecific anti-PD-L1 antibody, which may be a known antibody in the art or an antibody developed de novo. In some embodiments according to the present application, the PD-L1 binding moiety may be derived from a parental single-domain antibody (sdAb), such as a VHH antibody, which generally refers to an antibody consisting of a single variable domain. Like a whole antibody, a single-domain antibody is able to bind selectively to a specific antigen. In some other embodiments, the PD-L1 binding moiety may be derived from a heavy chain antibody, which is devoid of light chains.

The term “single variable domain” or “heavy chain variable region of a heavy chain antibody” is interchangeably used with the terms “VHH”, “VHH antibody”, “VHH domain”, “VHH antibody fragment”, “V_{HH}” or “Nanobody,” etc. V_{HH} molecules derived from Camelidae antibodies are among the smallest intact antigen-binding domains known (approximately 15 kDa, or 10 times smaller than a conventional IgG) and hence are well suited towards delivery to dense tissues and for accessing the limited space between macromolecules.

The parental VHH antibody as disclosed herein may be made by the skilled artisan according to methods known in the art or any future method. For example, VHHs may be obtained using methods known in the art such as by immunizing a camel and obtaining hybridoma's therefrom, or by cloning a library of VHHs of the invention using molecular biology techniques known in the art and subsequent selection by using phage display.

For instance, a VHH antibody can be obtained by immunization of llamas or alpacas with the desired antigen and subsequent isolation of the mRNA coding for heavy-chain antibodies. By reverse transcription and polymerase chain reaction, a gene library of single-domain antibodies containing several million clones is produced. Screening techniques like phage display and ribosome display help to identify the clones binding the antigen. One technique is phage display in which a library of (e.g., human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP phage display kit, catalog no. 240612). There also are other methods and reagents that can be used in generating and screening antibody display libraries (see, e.g., Barbas et al., Proc. Natl. Acad. Sci. USA 88:7978-7982 (1991)).

Humanization of the VHH antibody can be achieved via a number of well-established methods in the art, for example, amino acid sequences of VHH framework regions can be blasted against human germline V-gene database, and humanized VHH sequences can be generated by replacing human CDR sequences in the top hit with VHH CDR sequences using Kabat CDR definition. Further, certain residues in the framework region may be back mutated to maintain affinity.

In some embodiments, the PD-L1 antigen binding moiety comprises one or more CDRs selected from the group consisting of:

(i) a CDR1 comprising SEQ ID NO: 1 or an amino acid sequence that differs from SEQ ID NO: 1 by an amino acid addition, deletion or substitution of not more than 2 amino acids;

(ii) a CDR2 comprising SEQ ID NO: 2 or an amino acid sequence that differs from SEQ ID NO: 2 by an amino acid addition, deletion or substitution of not more than 2 amino acids; and

(iii) a CDR3 comprising SEQ ID NO: 3 or an amino acid sequence that differs from SEQ ID NO: 3 by an amino acid addition, deletion or substitution of not more than 2 amino acids.

In some embodiments, the PD-L1 antigen binding moiety is a VHH antibody that comprises (i) a CDR1 comprising or consisting of SEQ ID NO: 1; (ii) a CDR2 comprising or consisting of SEQ ID NO: 2; and (iii) a CDR3 comprising or consisting of SEQ ID NO: 3.

In some embodiments, the VHH of the PD-L1 antigen-binding moiety comprises: (i) the amino acid sequence of SEQ ID NO: 10; (ii) an amino acid sequence at least 85%, 90%, or 95% identical to SEQ ID NO: 10; or (iii) an amino acid sequence with addition, deletion and/or substitution of one or more (e.g. 10, 9, 8, 7, 6, 5, 4, 3, 2, 1) amino acid(s) compared with SEQ ID NO: 10.

The VEGF Antigen-Binding Moiety

Similarly, the VEGF antigen-binding moiety provided herein may be derived from a parental anti-VEGF monospecific antibody. In some embodiments according to the present application, the VEGF antigen-binding moiety is the Fab fragment of an anti-VEGF full antibody, i.e. comprising VH region and CH1 region in the heavy chain, and VL region and CL region in the light chain.

The anti-VEGF antibody which is used as the parental antibody may be a monoclonal antibody already known in the art (such as Bevacizumab) or which is de novo developed. Preferably, the anti-VEGF antibody is a fully human antibody or a humanized antibody.

In some embodiments, the VH region of the VEGF antigen-binding moiety comprises one or more heavy chain CDRs (HCDRs) selected from the group consisting of:

(i) a HCDR1 consisting of SEQ ID NO: 4 or a HCDR1 that differs in amino acid sequence from SEQ ID NO: 4 by an amino acid addition, deletion or substitution of not more than 2 amino acids;

(ii) a HCDR2 consisting of SEQ ID NO: 5 or a HCDR2 that differs in amino acid sequence from SEQ ID NO: 5 by an amino acid addition, deletion or substitution of not more than 2 amino acids; and

(iii) a HCDR3 consisting of SEQ ID NO: 6 or a HCDR3 that differs in amino acid sequence from SEQ ID NO: 6 by an amino acid addition, deletion or substitution of not more than 2 amino acids; and/or the VL region comprises one or more light chain CDRs (LCDRs) selected from the group consisting of:

(i) a LCDR1 consisting of SEQ ID NO: 7 or a LCDR1 that differs in amino acid sequence from SEQ ID NO: 7 by an amino acid addition, deletion or substitution of not more than 2 amino acids;

(ii) a LCDR2 consisting of SEQ ID NO: 8 or a LCDR2 that differs in amino acid sequence from SEQ ID NO: 8 by an amino acid addition, deletion or substitution of not more than 2 amino acids; and

(iii) a LCDR3 consisting of SEQ ID NO: 9 or a LCDR3 that differs in amino acid sequence from SEQ ID NO: 9 by an amino acid addition, deletion or substitution of not more than 2 amino acids.

In some embodiments, the VH comprises (i) a HCDR1 comprising or consisting of SEQ ID NO: 4; (ii) a HCDR2 comprising or consisting of SEQ ID NO: 5; and (iii) a HCDR3 comprising or consisting of SEQ ID NO: 6; and the VL comprises: (i) a LCDR1 comprising or consisting of SEQ ID NO: 7; (ii) a LCDR2 comprising or consisting of SEQ ID NO: 8; and (iii) a LCDR3 comprising or consisting of SEQ ID NO: 9.

In some embodiments, the VH of the VEGF antigen-binding moiety comprises: (i) the amino acid sequence of SEQ ID NO: 11; (ii) an amino acid sequence at least 85%, 90%, or 95% identical to SEQ ID NO: 11; or (iii) an amino acid sequence with addition, deletion and/or substitution of one or more (e.g. 10, 9, 8, 7, 6, 5, 4, 3, 2, 1) amino acids compared with SEQ ID NO: 11.

In some embodiments, the VL of the VEGF antigen-binding moiety comprises: (i) the amino acid sequence of SEQ ID NO: 12; (ii) an amino acid sequence at least 85%, 90%, or 95% identical to SEQ ID NO: 12; or (iii) an amino acid sequence with addition, deletion and/or substitution of one or more (e.g. 10, 9, 8, 7, 6, 5, 4, 3, 2, 1) amino acids compared with SEQ ID NO: 12.

The assignment of amino acids to each CDR may be in accordance with one of the numbering schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted.

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. MoI. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In other embodiments, the CDR amino acid sequences can be at least 90%, 91%, 92%, 93%, 94%, 95%, 96%, 97%, 98% or 99% identical to the respective sequences set forth above. In other embodiments, the amino acid sequences of the variable region can be at least 90%, 91%, 92%, 93%, 94%, 95%, 96%, 97%, 98% or 99% identical to the respective sequences set forth above.

Preferably, the CDRs of the isolated antibody or the antigen-binding portion thereof contain a conservative substitution of not more than 2 amino acids, or not more than 1 amino acid. The term “conservative substitution”, as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having (3-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

Generation of Bispecific Antibodies

For constructing of an anti-PD-L1/VEGF bispecific antibody, the PD-L1 antigen-binding moiety and the VEGF antigen-binding moiety as described above may be fused together in various formats. In certain embodiments, the PD-L1 antigen-binding moiety is fused to the N terminal of the VEGF antigen-binding moiety. When the PD-L1 antigen-binding moiety is a VHH, the single chain of the PD-L1 antigen-binding moiety may be operably linked to the heavy chain or light chain of the VEGF antigen-binding moiety, optionally via a linker. Preferably, the PD-L1 antigen-binding moiety is linked to the light chain of the VEGF antigen-binding moiety. The linker may be a peptide linker, such as comprising 1-4 copies of GGGGS (G4S). In one embodiment, the linker is (G4S)2.

The bispecific antibodies and antigen-binding portions provided herein can be made with any suitable methods known in the art. In a conventional approach, two immunoglobulin heavy chain-light chain pairs can be co-expressed in a host cell to produce bispecific antibodies in a recombinant way (see, for example, Milstein and Cuello, Nature, 305: 537 (1983)), followed by purification by affinity chromatography. The sequences encoding the antibody heavy chain variable domains for the two specificities can also be respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)).

Fc Region

In certain embodiments, the bispecific antibody comprises a Fc region that is operably linked to the VEGF antigen-binding moiety. The Fc region of the bispecific antibodies disclosed herein may be a human IgG Fc region. The IgG Fc region may be of any isotype, including, but not limited to, IgG1, IgG2, IgG3 or IgG4. In certain embodiments, the Fc region is of the IgG1 isotype.

In the context of bispecific antibodies of the present disclosure, the Fc region may comprise one or more amino acid changes (e.g., insertions, deletions or substitutions) as compared to the specified chimeric version of the Fc region. For example, the disclosure includes bispecific antigen-binding molecules comprising one or more modifications in the Fc region that results in a modified Fc region having a modified binding interaction between Fc and FcRn or FcγR.

The “EU numbering system” or “EU index” is generally used when referring to a residue in an immunoglobulin heavy chain constant region (e.g., the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

In certain embodiments, the Fc region is operably linked to the VEGF binding moiety via a hinge region. Optionally, the hinge region may be derived from human IgG1, IgG2 or IgG4. In certain embodiments, the hinge region is derived from human IgG1.

Nucleic Acid Molecules Encoding Antibodies of the Disclosure

In some aspects, the disclosure is directed to an isolated nucleic acid molecule, comprising a nucleic acid sequence encoding the bispecific antibody or the antigen-binding portion as disclosed herein. For example, the nucleic acid sequence may encode a heavy chain and/or a light chain of the bispecific antibody. Alternatively, the nucleic acid sequence may encode the PD-L1 antigen-binding moiety, or a heavy chain or a light chain variable region of the VEGF antigen-binding moiety. The nucleic acid sequence may further encode the Fc region of the bispecific antibody.

In some aspects, the disclosure is directed to a vector comprising the nucleic acid sequence as disclosed herein. In a further embodiment, the expression vector further comprises a nucleotide sequence encoding the constant region of a bispecific antibody, e.g. a humanized bispecific antibody.

A vector in the context of the present disclosure may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, a PD-L1 or a VEGF antibody-encoding nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaPO4-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the anti-PD-L1 antibody and/or anti-VEGF antibody in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison Wis.) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine synthetase as a selectable marker, such as the vectors described in Bebbington (1992) Biotechnology (NY) 10: 169-175.

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In a further aspect, the disclosure relates to a host cell comprising the vector specified herein above. Thus, the present disclosure also relates to a recombinant eukaryotic or prokaryotic host cell which produces a bispecific antibody of the present disclosure, such as a transfectoma.

The PD-L1-specific antibody may be expressed in a recombinant eukaryotic or prokaryotic host cell, such as a transfectoma, which produces an antibody of the disclosure as defined herein or a bispecific antibody of the disclosure as defined herein. The VEGF-specific antibody may likewise be expressed in a recombinant eukaryotic or prokaryotic host cell, such as a transfectoma, which produces an antibody of the disclosure as defined herein or a bispecific antibody of the disclosure as defined herein.

Examples of host cells include yeast, bacterial, plant and mammalian cells, such as CHO, CHO-S, HEK, HEK293, HEK-293F, Expi293F, PER.C6 or NSO cells or lymphocytic cells. For example, in one embodiment, the host cell may comprise a first and second nucleic acid construct stably integrated into the cellular genome. In another embodiment, the present disclosure provides a cell comprising a non-integrated nucleic acid, such as a plasmid, cosmid, phagemid, or linear expression element, which comprises a first and second nucleic acid construct as specified above.

In a further aspect, the disclosure relates to a transgenic non-human animal or plant comprising nucleic acids encoding one or two sets of a human heavy chain and a human light chain, wherein the animal or plant produces a bispecific antibody of the disclosure.

In a further aspect, the disclosure relates to a hybridoma which produces an antibody for use in a bispecific antibody of the disclosure as defined herein.

In one aspect, the disclosure relates to an expression vector comprising:

(i) a nucleic acid sequence encoding the PD-L1 antigen-binding moiety;

(ii) a nucleic acid sequence encoding the heavy chain and/or the light chain of the VEGF antigen-binding moiety;

(iii) a nucleic acid sequence encoding a Fc region; or

(iv) a nucleic acid sequence encoding the heavy chain or the light chain of the bispecific antibody.

In one aspect, the disclosure relates to a nucleic acid construct encoding one or more amino acid sequences set out in the sequence listing.

In one aspect, the disclosure relates to a method for producing a bispecific antibody according to any one of the embodiments as disclosed herein, comprising the steps of culturing a host cell as disclosed herein comprising an expression vector or more than one expression vectors as disclosed herein expressing the bispecific antibody as disclosed herein and purifying said antibody from the culture media. In one aspect, the disclosure relates to a host cell comprising an expression vector as defined above. In one embodiment, the host cell is a recombinant eukaryotic, recombinant prokaryotic, or recombinant microbial host cell.

Pharmaceutical Compositions

In some aspects, the disclosure is directed to a pharmaceutical composition comprising at least one bispecific antibody or antigen-binding portion thereof as disclosed herein and a pharmaceutically acceptable carrier.

Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-PD-L1/anti-VEGF bispecific antibody enhances the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, in a solvent containing an antibody or an antigen-binding fragment of the present disclosure discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight per dose.

In any event, the antibody or the antigen binding portion thereof of the disclosure is preferably administered as needed to a subject in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the instant disclosure will comprise multiple doses of the selected drug product over a period of weeks or months. More specifically, the antibody or the antigen-binding portion thereof of the instant disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentrations of the antibody or the antigen binding portion thereof will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

Applications of the Disclosure

In some aspects, the present disclosure provides a method of treating a disorder in a subject, which comprises administering to the subject (for example, a human) in need of treatment a therapeutically effective amount of the antibody or antigen-binding portion thereof as disclosed herein. For example, the disorder is a cancer.

A variety of cancers where PD-L1 and/or VEGF is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer.

In some embodiments, examples of cancer include but not limited to B-cell cancers, including B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliierative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some other embodiments, the disorder is an autoimmune disease. Examples of autoimmune diseases that may be treated with the antibody or antigen-binding portion thereof include autoimmune encephalomyelitis, lupus erythematosus, and rheumatoid arthritis. The antibody or the antigen-binding portion thereof may also be used to treat or prevent infectious disease, inflammatory disease (such as allergic asthma) and chronic graft-versus-host disease.

Combined Use with Chemotherapies

The antibody or the antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the instant disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic Rnases, such as extracellular pancreatic Rnases; Dnase I, including fragments and/or variants thereof).

For the purposes of the instant disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMIYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LUIRTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of the antibody or the antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed composition is used for treating the neoplastic disease condition of choice.

The present disclosure also provides kits for producing single-dose or multi-dose administration units of antibodies and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed antibodies, either in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the antibodies and, optionally, one or more anti-cancer agents in the same or different containers. The kits may also contain other pharmaceutically acceptable formulations, either for diagnosis or combined therapy. For example, in addition to the antibody or the antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the antibodies and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

Sequence Listing Summary

Appended to the instant application is a sequence list comprising a number of amino acid sequences. The following Table A provides a summary of the included sequences.

One illustrative antibody as disclosed herein, which is an anti-VEGF/anti-PD-L1 bispecific antibody, is designated as W3256-U15T2.G6-1.uIgG1 (abbreviated as “W3256” throughout the disclosure).

TABLE A

| SEQ | |
|---|---|
| ID | |
| NO. | Description |
| 1 | Amino acid sequence of CDR1 of PD-L1 binding moiety |
| 2 | Amino acid sequence of CDR2 of PD-L1 binding moiety |
| 3 | Amino acid sequence of CDR3 of PD-Ll binding moiety |
| 4 | Amino acid sequence of Heavy chain CDR1 of VEGF |
| | binding moiety |
| 5 | Amino acid sequence of Heavy chain CDR2 of VEGF |
| | binding moiety |
| 6 | Amino acid sequence of Heavy chain CDR3 of VEGF |
| | binding moiety |
| 7 | Amino acid sequence of Light chain CDR1 of VEGF |
| | binding moiety |
| 8 | Amino acid sequence of Light chain CDR2 of VEGF |
| | binding moiety |
| 9 | Amino acid sequence of Light chain CDR3 of VEGF |
| | binding moiety |
| 10 | Amino acid sequence of VHH of PD-L1 binding moiety |
| 11 | Amino acid sequence of VH of VEGF binding moiety |
| 12 | Amino acid sequence of VL of VEGF binding moiety |
| 13 | Amino acid sequence of the heavy chain of W3256 |
| 14 | Amino acid sequence of the light chain of W3256 |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Benchmark Antibodies and Cell Lines

### 1.1 Preparation of Materials

Information on the commercially available materials used in the examples are provided in Table 1.

TABLE 1

| Materials | Vendor | Cat. |
|---|---|---|
| Expi293 Expression System | Thermo Fisher Scientific | A14635 |
| Protein A column | GE healthcare | 17-5438-02 |
| Ni column | GE healthcare | 17-5247-01 |
| HPLC-SEC | TOSOH | 0008541 |
| NuPAGE4%-12% Bis-Tris Gel | Invitrogen | NP0322BOX |
| Human VEGF | Sino Biological | 11066-HNAB |
| Mouse VEGF | Sino Biological | 50159-MNAB |
| Human VEGF, Biotin labeled | ACRO Biosystems | VE5-H8210 |
| Human VEGFR1, hFc tag | Sino Biological | 10136-H02H |
| Mouse VEGFR1, hFc and his tag | R&D | 471-F1 |
| Human PD-L1, His tag | Sino Biological | 10084-H08H |
| Cynomolgus PD-L1, His tag | Sino Biological | 90251-C08H |
| Mouse PD-L1, His tag | Sino Biological | 50010-M08H |
| Goat-anti-human IgG Fc-HRP | Bethyl | A80-304P |
| antibody | | |
| RPMI1640 | Gibco | 22400-089 |
| Ficoll-Paque | Stemcell | 07851 |
| Monocyte enrichment kit | Miltenyi Biotec | 130-050-201 |
| CD4⁺ T cell enrichment kit | Stemcell | 19052 |
| Goat x-human IgG Fc FITC | Bethyl | A80-304F |
| Conjugated | | |
| Recombinant human GM-CSF | R&D | 215-GM-010 |
| Recombinant human IL-4 | R&D | 204-IL-010 |
| LPS | Sigma | L5418 |
| Recombinant human IL-2 standard | R&D | 202-IL-050 |
| Recombinant human IFN γ standard | PeproTech | 300-02 |
| anti-hIL-2 purified Mouse | R&D | MAB602 |
| Monoclonal IgG2A Clone 5355 | | |
| anti-hIL-2 Biotinylated Goat IgG | R&D | BAF202 |
| Human IFNγ Mab clone 2G1 | Thermo prod | M700A |
| Human IFNγ Mab Biotin-Labeled | Thermo prod | M701B |
| Streptavidin HRP | Invitrogen | SNN1004 |
| H3-thymidine and MicroScint | Perkin Elmer | NET027001MC |
| Human serum complement | Quidel | A113 |
| Fetal bovine serum (FBS) | ExCell Bio | FND500 |
| penicillin-streptomycin (PS) | Invitrogen | SV30010 |
| CellTiter-Glo Luminescent Cell | Promega | G7573 |
| Viability Assay kit | | |

1.2 Antigen Preparation

DNA sequences encoding the sequences of human VEGF (Uniport No.: P15692), mouse VEGF (Uniport No.: Q00731), and the extracellular domain sequences of human PD-L1 (Uniport No.: Q9NZQ7), mouse PD-L1 (Uniport No.: Q9EP73), human PD-1 (Uniport No.: Q15116), and mouse PD-1 (Uniport No.: Q02242) were synthesized in Sangon Biothech (Shanghai, China), and then subcloned into modified pcDNA3.3 expression vectors with different tags (such as 6×his, human Fc, or mouse Fc) at C-terminus.

Expi293 cells (Thermo Fisher Scientific, A14527) were transfected with the purified expression vector. Cells were cultured for 5 days and supernatant was collected for protein purification using Ni-NTA column (GE Healthcare, 175248), Protein A column (GE Healthcare, 175438) or Protein G column (GE Healthcare, 170618). The obtained human VEGF, human PD-L1, mouse PD-L1, human PD-1 and mouse PD-1 were QC'ed by SDS-PAGE and SEC, and then stored at −80° C.

1.3 Production of Benchmark Antibodies (BMK Abs)

DNA sequence encoding the fragment of anti-VEGF antibodies, Bevacizumab (named as WBP325-BMK3 or WBP325-BMK3.uIgG1, sequence from Drug Bank, Drug Bank No.: DB00112) was synthesized in Sangon Biothech (Shanghai, China) or Genewiz (Suzhou, China), and then subcloned into modified pcDNA3.3 expression vectors with Fc region of human IgG1.

Anti-PD-L1 VHH antibody (W3156-AP3R2-1A3-z12) was generated by immunizing Alpacas with extracellular domain of human PD-L1 and mouse PD-L1 alternately, and PBMCs were isolated for phage library construction. After panning, screening and sequencing, one unique positive VHH fragment was identified (SEQ ID No: 4 as disclosed in patent No: PCT/CN2020/117351).

Atezolizumab (named as W315-BMK8.uIgG1K(RKNA) or abbreviated as W315-BMK8), the anti-PD-L1 antibody developed by Roche, was used as a control antibody.

For producing WBP325-BMK3, the plasmid containing recombination VH and VL gene were co-transfected into Expi293 cells. Cells were cultured for 5 days and supernatant was collected for protein purification using Protein A column (GE Healthcare, 175438) or Protein G column (GE Healthcare, 170618). The obtained antibodies were analyzed by SDS-PAGE and SEC, and then stored at −80° C.

### 1.4 Establishment of Stable Cell Lines/Cell Pool

### PD-L1-Expressing Cell Lines

Using Lipofectamine 2000 (Thermo Fisher Scientific, 11668019) or PlasFect (Bioline, 46026), CHO-K1 or 293F cells were transfected with the expression vector containing gene encoding full length human PD-L1 or mouse PD-L1 or cynomolgus monkey PD-L1. Cells were cultured in medium containing proper selection marker. Human PD-L1 high expression stable cell line (WBP315.CHO-K1.hPro1.C11), mouse PD-L1 high expression stable cell line (WBP315.293F.mPro1.C1) and cynomolgus monkey PD-L1 high expression stable cell line (WBP315.293F.cPro1.2A) were obtained by limiting dilution.

The genes of human PD-L1 or mouse PD-L1 or cynomolgus monkey PD-L1 were respectively inserted into expression vector pcDNA 3.3. The plasmids were then transfected to CHO-K1 cells or 293 cells, respectively. Briefly, for CHO-K1 cells, one day prior to transfection, 5×10⁵ CHO-K1 cells were plated into one well of 6-well tissue culture plate and incubated at 5% CO₂ and 37° C. The cells were fed with 3 ml of fresh non-selective media (F12-K, 10% FBS). Transfection reagents were prepared in a 1.5 ml tube, including 4 μg of DNA mixed with 10 μg of Lipofectamine 2000 to make the final volume 200 μl in Opti-MEM medium. The solution in the tube pipette was added to the cells drop by drop. 6-8 hours after transfection, cells were washed with PBS and feed with 3 ml of fresh non-selective media. Expressing cells were harvested with trypsin 24-48 hours post-transfection and plated to T75 flask in selective media (F12-K, 10% FBS, 10 μg/ml Blasticidin). For 293F cells, 20 μg of DNA mixed with 50 μl of PlasFect to make the final volume 200 μl in Opti-MEM medium. The mixture was added into 20 ml (1×10⁶/ml) suspended 293F cells, and cultured in Freestyle293 medium in 125 ml-flask. 48 hours post-transfection, blasticidin was added as selection marker. After two or three passages of selection, the cells were detected by anti-PD-L1 antibody. Stable single cell clones were isolated by limiting dilution and screened by FACS using anti-PD-L1 antibodies.

### Target-Expressing Cell Lines

Human Umbilical Vein Endothelial Cell (HUVEC) was purchased from ScienceCell (Cat: 8000) and cultured in endothelial cell medium (ECM, ScienceCell, Cat: 1001) containing basic medium, 5% FBS, 1% endothelial cell growth factor (ECGS, ScienceCell, 1052). The cells were cultured in an incubator with 37° C. and 5% CO₂. For long term storage, the cells were frozen in complete growth medium supplemented with 5% (v/v) DMSO and stored in liquid nitrogen vapor phase.

### Example 2

### Generation of PD-L1/VEGF Bispecific Antibodies

### 2.1 Construction of Expression Vectors

DNA sequence encoding the VHH antibody W3156-AP3R2-1A3-z12 linked by flexibly G4S linker was put on the N-terminal of light chain of WBP325-BMK3 (Bevacizumab). The heavy chain was constructed using the sequence same as the sequence of WBP325-BMK3 (Bevacizumab). The recombinant DNA sequence were cloned into modified pcDNA3.3 expression vector, respectively. The constructed antibody was named as W3256-U15T2.G6-1.uIgG1 (abbreviated as “W3256” throughout the disclosure).

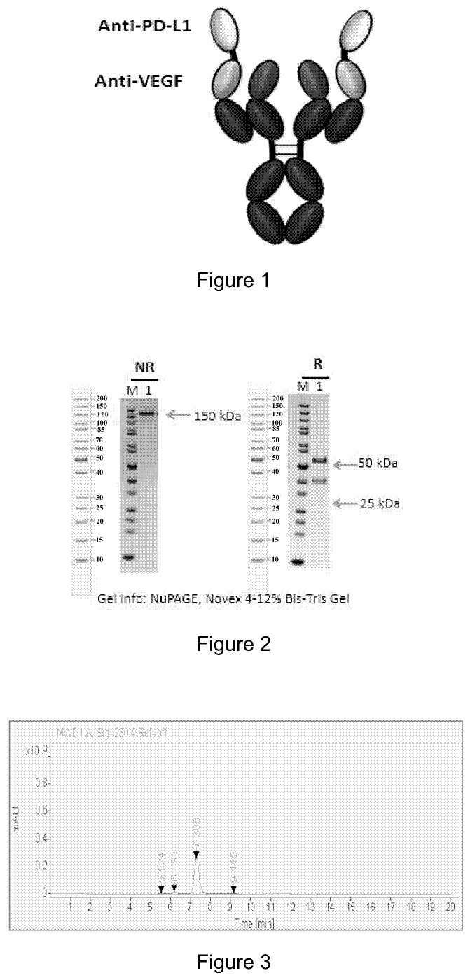

As described above, the heavy chain of W3256-U15T2.G6-1.uIgG1 comprises the variable heavy chain region of WBP325-BMK3 (Bevacizumab) and constant heavy chain region (CH1-CH3) of human IgG1. The light chain of W3256-U15T2.G6-1.uIgG1 is composed of the variable light chain region of WBP325-BMK3 (Bevacizumab) and constant light chain region (CL) of human IgG1 with VHH antibody W3156-AP3R2-1A3-z12 on the N-terminal, as shown in FIG. 1. The specific sequences of W3256 antibody are listed in Tables 2-4 below.

TABLE 2

| | | | |
|---|---|---|---|
| Anti- | HCDR1 | HCDR2 | HCDR3 |
| VEGF | SEQ ID NO: 4 | SEQ ID NO: 5 | SEQ ID NO: 6 |
| | GYTFTNYGMN | WINTYTGEPTYAADFKR | YPHYYGSSHWYFDV |
| | LCDR1 | LCDR2 | LCDR3 |
| | SEQ ID NO: 7 | SEQ ID NO: 8 | SEQ ID NO: 9 |
| | SASQDISNYLN | FTSSLHS | QQYSTVPWT |
| Anti- | CDR1 | CDR2 | CDR3 |
| PD-L1 | SEQ ID NO: 1 | SEQ ID NO: 2 | SEQ ID NO: 3 |
| VHH | GHFSNLAVN | GILWSGGSTFYADSVKG | GTN |

TABLE 3
Amino acid sequence of VH of VEGF binding moiety:
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSS
(SEQ ID NO: 11)
Amino acid sequence of VL of VEGF binding moiety:
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIK
(SEQ ID NO: 12)
Amino acid sequence of VHH of PD-L1 binding
moiety:
EVQLVESGGGLVQPGGSLRLSCAASGHFSNLAVNWFRQAPGKERELVAG
ILWSGGSTFYADSVKGRFTISRGNAENMLYLQMNSLRAEDTAVYYCNTG
TNWGQGTLVTVSS
(SEQ ID NO: 10)

TABLE 4
Amino acid sequence of the heavy chain of
W3256-U15T2.G6-1.uIgG1:
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSSASTKGPSVFPLAPSSKSTSGGTAA
LGCLVKDYFPEPVTVSWNSGALTSGVHTFPAVLQSSGLYSLSSWTVPSS
SLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHY
TQKSLSLSPG
(SEQ ID NO: 13)
Amino acid sequence of the light chain of
W3256-U15T2.G6-1.uIgG1:
EVQLVESGGGLVQPGGSLRLSCAASGHFSNLAVNWFRQAPGKERELVAG
ILWSGGSTFYADSVKGRFTISRGNAENMLYLQMNSLRAEDTAVYYCNTG
TNWGQGTLVTVSSGGGGSGGGGSDIQMTQSPSSLSASVGDRVTITCSAS
QDISNYLNWYQQKPGKAPKVLIYFTSSLHSGVPSRFSGSGSGTDFTLTI
SSLQPEDFATYYCQQYSTVPWTFGQGTKVEIKRTVAAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGEC
(SEQ ID NO: 14)

### 2.2 Transfection, Expression and Purification

Heavy chain and light chain expression plasmids were co-transfected into Expi293 cells using Expi293 expression system kit (ThermoFisher-A14635) according to the manufacturer's instructions. 5 days after transfection, the supernatants were collected and used for protein purification using Protein A column (GE Healthcare-17543802). Antibody concentration was measured by NanoDrop. The purity of proteins was evaluated by SDS-PAGE and HPLC-SEC. Bispecific antibodies, including W3256-U15T2.G6-1.uIgG1 was obtained after expression and purification.

### 2.3 Production of Bispecific Antibody for In Vivo Studies (Including Endotoxin Control and Test)

W3256-U15T2.G6-1.uIgG1 expression plasmids were transfected into Expi293 cells using Expi293 expression system kit (ThermoFisher-A14635) according to the manufacturer's instructions. Five days after transfection, the supernatants were collected and used for protein purification using Protein A column (GE Healthcare-17543802) under endotoxin controlled condition. The low endotoxin level (less than 10 EU/mg) was confirmed by using endotoxin detection kit (GenScript-L00350).

Antibody concentration was measured by NanoDrop, and the yield of W3256-U15T2.G6-1.uIgG1 is 44.8 mg/l. The purity of proteins was evaluated by SDS-PAGE (FIG. 2) and HPLC-SEC (FIG. 3). According to HPLC-SEC, the purity of W3256-U15T2.G6-1.uIgG1 is 93.43% after Protein A one-step purification.

### Example 3

### In Vitro Characterization of the Bispecific Antibodies

### 3.1 Differential Scanning Fluorimetry (DSF)

A DSF assay was performed using 7500 Fast Real-Time PCR system (Applied Biosystems). Briefly, 19 μl of bispecific antibody solution was mixed with 1 μl of 62.5×SYPRO Orange solution (ThermoFisher-S6650) and added to a 96 well plate. The plate was heated from 26° C. to 95° C. at a rate of 2° C./min and the resulting fluorescence data was collected. The data was analyzed automatically by its operation software and Th was calculated by taking the maximal value of negative derivative of the resulting fluorescence data with respect to temperature. Tₒₙ can be roughly determined as the temperature of negative derivative plot beginning to decrease from a pre-transition baseline.

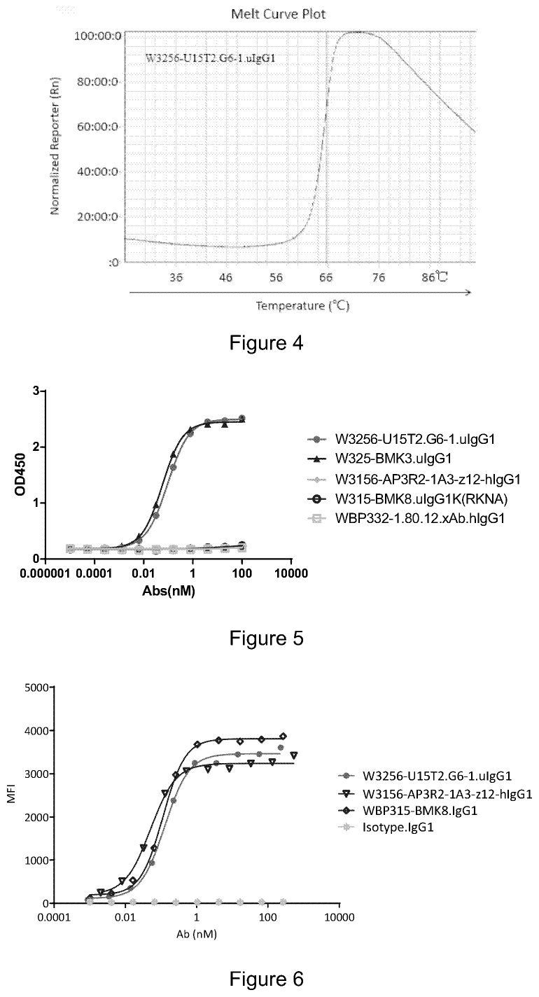

As shown in FIG. 4, the Tₕ1 value of the W3256 antibody is 65.7° C.

### 3.2 Human/Cynomolgus Monkey VEGF-Binding (ELISA)

The binding of the antibodies to human VEGF antigen (WBP325-hPro1, Sino Biological, 11066-HNAB) were tested by binding ELISA assay. Since the amino acid sequence of cynomolgus monkey VEGF is the same as human VEGF, the binding result represents both human VEGF binding and cyno VEGF binding. Briefly, 96-well ELISA plates (Nunc MaxiSorp, ThermoFisher, 442404) were coated overnight at 4° C. with 0.25 μg/ml human VEGF in Carbonate-bicarbonate buffer (20 mM Na₂CO₃, 180 mM NaHCO₃, pH9.2). After a 1 hour blocking step with 2% (w/v) bovine serum albumin (Pierce) dissolved in PBS, serial dilutions of the W3256-U15T2.G6-1. UIgG1 are incubated on the plates for 2 hours at room temperature. Following the incubation, plates are washed three times with 300 μL per well of PBS containing 0.5% (v/v) Tween 20. 100 ng/ml Goat-anti-human IgG Fc-HRP (Bethyl, #A80-304P) is added and incubated on the plates for 1 hour at room temperature. After washing six times, Tetramethylbenzidine (TMB) Substrate (Sigma-860336-5G) is added for the detection. The reaction is stopped after approximate 8 minutes through the addition of 100 μl per well of 2 M HCl. The absorbance of the wells is measured at 450 nm with a multiwell plate reader (SpectraMax® M5^{e}).

W3256 shows comparable binding ability to WBP325-BMK3.uIgG1 on human VEGF with EC₅₀ of ˜0.095 nM (FIG. 5).

### 3.3 Human PD-L1 Binding (FACS)

Engineered human PD-L1 expressing cells (WBP315.CHO-K1.hPro1.C11) were seeded at 1×10⁵ cells/well in U-bottom 96-well plates. Serial dilutions of W3256-U15T2.G6-1. UIgG1 was added to the cells. Plates were incubated at 4° C. for 1 hour. After wash, FITC-labeled goat anti-human IgG (Jackson ImmunoResearch, 109-095-008) was added to each well and the plates were incubated at 4° C. for 1 hour. The binding of the antibodies onto the cells was tested by flow cytometry and the mean fluorescence intensity (MFI) was analyzed by FlowJo.

W3256 also shows comparable binding ability to W3156-AP3R2-1A3-z12-hIgG1 on human PD-L1 with EC₅₀ of ˜0.128 nM (FIG. 6).

### 3.4 Human VEGF/Human PD-L1 Dual Binding (ELISA)

In order to test whether the bispecific antibodies could bind to both VEGF and PD-L1, an ELISA assay was developed as below. A 96-well ELISA plate (Nunc MaxiSorp, ThermoFisher) was coated overnight at 4° C. with 1 μg/ml antigen-1 (VEGF, WBP325-hPro1, Sino Biological) or 1 μg/ml antigen-2 (PD-L1.ECD.mFc, W3153-hPro1.ECD.mFc) in carbonate-bicarbonate buffer. After a 1 hour blocking step, serial dilutions of W3256-U15T2.G6-1. UIgG1 in casein buffer are incubated on the plates for 1 hour at room temperature. Following the incubation, plates are washed three times with 300 μL per well of PBS containing 0.5% (v/v) Tween 20. 0.5 μg/ml antigen-2.Biotin (PD-L1-ECD, WBP315-hPro1.ECD.mFc) or 0.5 μg/ml antigen-1.Biotin (VEGF, WBP325-hPro1. his) was added to plates and incubated for 1 hour. After washing the plates three times, HRP labeled secondary detection antibody is added and incubated on the plates for 1 hour at room temperature. After washing the plates six times, Tetramethylbenzidine (TMB) Substrate (Sigma-860336-5G) is added for the detection. The reaction is stopped after approximate 10 minutes through the addition of 100 μL per well of 2 M HCl. The absorbance of the wells is measured at 450 nm with a multiwell plate reader (SpectraMax® M5^{e}).

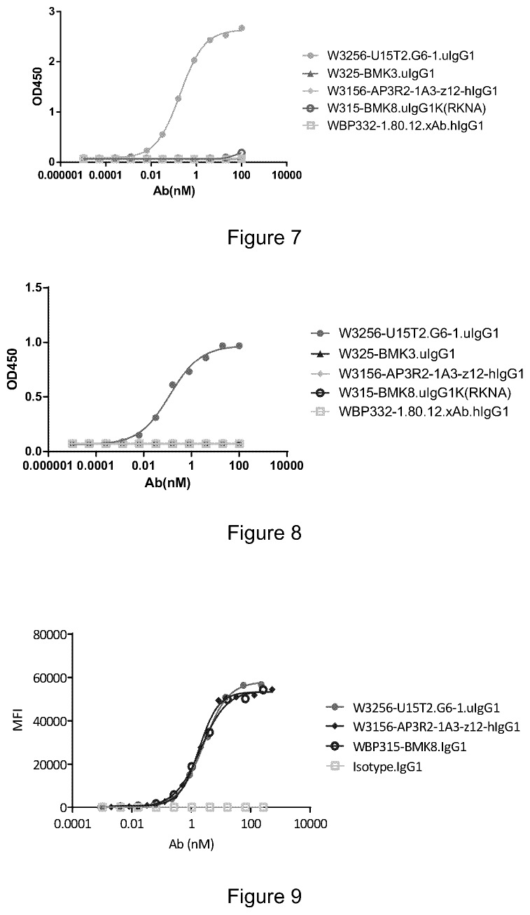

The results show that the binding of VEGF to W3256 didn't affect the subsequent binding of PD-L1 (FIG. 7), and vice versa (FIG. 8).

### 3.5 Cross Species Binding (FACS/ELISA)

Cyno PD-L1 Binding (FACS)

Engineered cynomolgus monkey PD-L1 expressing cells (WBP315.293F.cPro1.2A) were seeded at 1×10⁵ cells/well in U-bottom 96-well plates. Serial dilutions of W3256-U15T2.G6-1. UIgG1 was added to the cells. Plates were incubated at 4° C. for 1 hour. After wash, FITC-labeled goat anti-human IgG (Jackson ImmunoResearch, 109-095-008) was added to each well and the plates were incubated at 4° C. for 1 hour. The binding of the antibodies onto the cells was tested by flow cytometry and the mean fluorescence intensity (MFI) was analyzed by FlowJo.

Mouse VEGF Binding (ELISA)

The binding of the antibodies to the mouse VEGF antigen (WBP325-mPro1, Sino Biological, 50159-MNAB) were tested by ELISA assay same as human VEGF binding described above, except the coating protein is 100 μL of 0.25 μg/mL Mouse VEGF.

Mouse PD-L1 Binding (FACS)

Engineered mouse PD-L1 expressing cells (WBP315.293F.mPro1.C1) were seeded at 1×10⁵ cells/well in U-bottom 96-well plates. Serial dilutions of the different antibodies were added to the cells. Plates were incubated at 4° C. for 1 hour. After wash, FITC-labeled goat anti-human IgG (Jackson ImmunoResearch, 109-095-008) was added to each well and the plates were incubated at 4° C. for 1 hour. The binding of W3256-U15T2.G6-1. UIgG1 onto the cells was tested by flow cytometry and the mean fluorescence intensity (MFI) was analyzed by FlowJo.

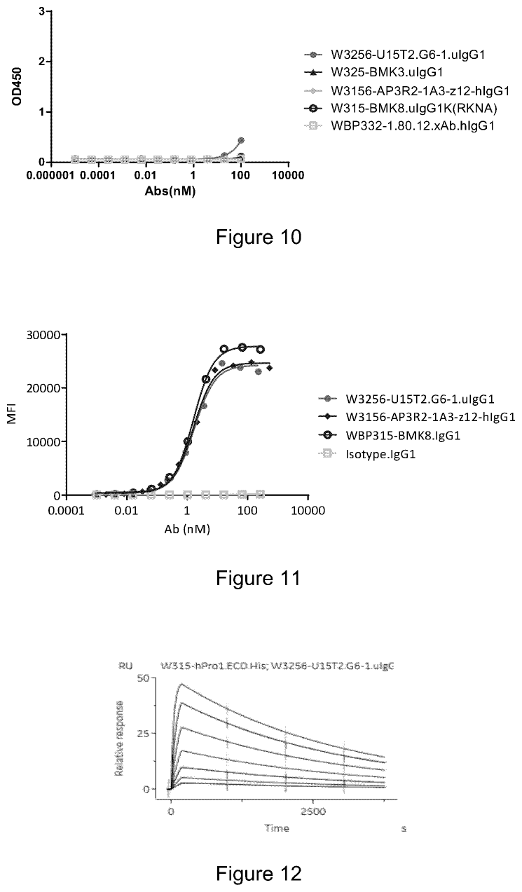

The amino acid sequence of cynomolgus monkey VEGF is the same with human VEGF, therefore, W3256 also has binding activity to cynomolgus VEGF. Cross species binding activity of W3256 to cynomolgus PD-L1, mouse VEGF, and mouse PD-L1 were also evaluated. Their binding activity to VEGF or PD-L1 were detected as shown in FIG. 9, FIG. 10 and FIG. 11. W3256 shows comparable binding ability to the parental antibodies on cynomolgus PD-L1 with EC₅₀ of ˜2.618 nM (FIG. 9). W3256 could not bind to mouse VEGF (FIG. 10). W3256 also shows comparable binding ability to the parental antibodies on mouse PD-L1 with EC₅₀ of ˜1.687 nM (FIG. 11).

### 3.6 Affinity of Binding to VEGF and PD-L1 (SPR)

SPR technology was used to measure the on-rate constant (ka) and off-rate constant (kd) of the antibodies to VEGF or ECD of PD-L1. The affinity constant (KD) was consequently determined.

Biacore T200, Series S Sensor Chip CM5, Amine Coupling Kit, and 10×HBS-EP were purchased from GE Healthcare. The antibody was captured onto the anti-human Fc IgG (Jackson, 109-005-098) surface immobilized on CM5-biosensor chip (GE Healthcare Inc.). The assay was performed at 25° C. with HBS-EP+ buffer (GE Healthcare Inc.) as running and dilution buffer. Serially diluted PD-L1 antigen or VEGF antigen (W315-hPro1.ECD.his or W325-hPro1.his) and running buffer were injected for an association phase and followed dissociation phase detection. Regeneration of the chip surface was reached by an injection of 10 mM Glycine, pH 1.5.

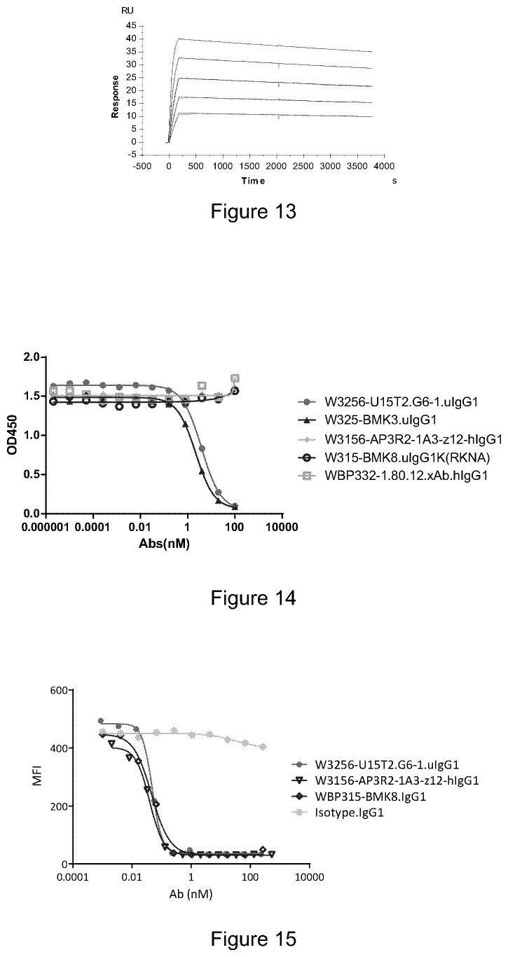

The affinity constant (KD) of W3256 was measured based on SPR technology. The on-rate constant (ka) and off-rate constant (kd) were also measured in the mean time. Final data of each interaction was deducted from reference channel and buffer channel data. The experimental data was analysed as shown in FIG. 12 and FIG. 13. The Kinetic affinity results of antibodies were listed in Table 5.

TABLE 5
Kinetic affinity results of antibodies

| Analyte | Ligand | ka1 (1/Ms) | kd1 (1/s) | KD1 (M) |
|---|---|---|---|---|
| W315- | W3256 | 2.14E+06 | 3.33E−04 | 1.55E−10 |
| hPro1.ECD.his | W3156-AP3R2-1A3-z12-hIgG1 | 1.82E+06 | 2.91E−04 | 1.60E−10 |
| (PD-L1) | W315-BMK8.uIgG1K(RKNA) | 1.53E+06 | 9.29E−05 | 6.08E−11 |
| W325- | W3256 | 2.38E+06 | 3.64E−05 | 1.53E−11 |
| hPro1.ECD.his | W325-BMK3.uIgG1 | 3.94E+06 | 3.69E−05 | 9.36E−12 |
| (VEGF) | | | | |

### 3.7 Ligand Competition Assay (ELISA)

### Human VEGFR1 Competition Assay

Inhibition of VEGF binding to the human VEGF receptor 1 (W325-hpro1R1.ECD.hFc, Sino Biological, 10136-H02H) was determined by competitive ELISA. 96-well ELISA plates (Nunc MaxiSorp, ThermoFisher, 442404) were coated overnight at 4° C. with 2 μg/mL W325-hpro1R1.ECD.His in Carbonate-bicarbonate buffer (20 mM Na₂CO₃, 180 mM NaHCO₃, PH9.2). All wells were washed three time with 300 μL per well of PBS/0.5% Tween-20 (v/v) and all the following wash steps in the assay were performed the same. The wells were then blocked for one hour with 200 μL per well of 50% Casein (Thermo SCIENTIFIC, 37528), then after wash steps, serial diluted antibody and 0.02 μg/ml biotin-labeled human VEGF (W325-hPro1.Biotin, ACRO Biosystems, VE5-H8210) mixture were added to the wells and incubated at 25° C. for two hours. Plates were washed three times prior to the addition of the peroxidase linked streptavidin (Jackson, 016-030-084) 1: 10000 diluted in PBS/50% Casein. Plates were incubated at 25° C. one hour before washing six times like before. 100 μL/well Tetramethylbenzidine (TMB) Substrate (Sigma, 860336) was added to all wells for 8 minutes before stopping the reaction with 100 μL 2M HCl. The extent of human VEGF-biotin binding to W325-hpro1R1.ECD.hFc was determined by measuring the OD₄₅₀ with a multiwell plate reader (SpectraMax® M5^{e}). IC₅₀ values of binding were obtained by the four-parameter non-linear regression analysis using GraphPad Prism 5 software.

W3256 shows comparable competition ability to the parental antibodies against hVEGFR1, binding to VEGF with IC₅₀ of ˜3.86 nM (FIG. 14).

### Mouse VEGFR1 Competition Assay

The effect of the antibodies block VEGF binds to the mouse VEGF receptor 1 (W325-mpro1R1.ECD.hFc.his (R&D, 471-F1) was assessed by competitive ELISA. The mouse VEGFR1 competition ELISA method is similar with the human VEGFR1 competition ELISA, except the protein pre-coated in 96-well ELISA plate is 2 μg/ml of W325-mpro1R1.ECD.hFc.his, the blocking buffer is PBS/2% BSA, and the concentration of W325-hPro1.Biotin is 0.44 μg/ml.

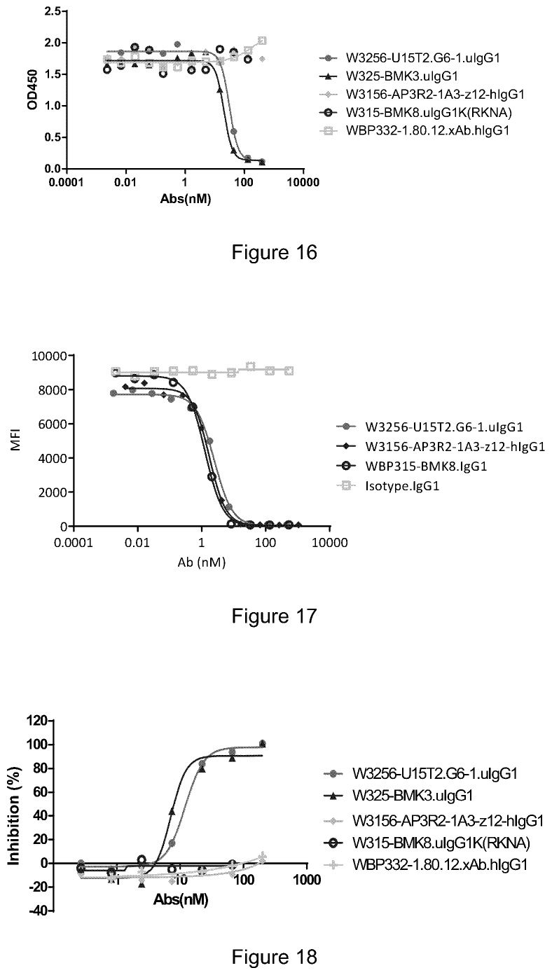

W3256 shows comparable competition ability to the parental antibodies against mVEGFR1 (FIG. 16), binding to VEGF with IC₅₀ of ˜31 nM.

### Human PD-1 Competition Assay

Competition of W3256 to human PD-1 was measured by FACS-based competition assay. Human PD-L1 expressing CHO-K1 cells (WBP315.CHO-K1.hPro1.C11) were seeded at 1×10⁵ cells/well in 96-well U-bottom plates. Serial dilutions of testing antibody was premixed with 5 μg/ml of mouse Fc tagged human PD-1, and then added to the cells and incubated for 1 hour at 4° C. After washing, PE-labeled anti-mouse IgG was added and incubated with cells at 4° C. for 45 minutes. Cells were washed twice and MFI of the cells was measured by a flow cytometer and analyzed by FlowJo.

W3256 shows comparable competition ability to the parental antibodies and block hPD-1 (FIG. 15) binding to PD-L1 with IC₅₀ of ˜0.048 nM.

### Mouse PD-1 Competition Assay

Mouse PD-L1 expressing CHO-K1 cells (WBP315.293F.mPro1.C1) were seeded at 1×10⁵ cells/well in 96-well U-bottom plates. Serial dilutions of testing antibodies were premixed with 5 μg/ml of mouse Fc tagged murine PD-1, and then added to the cells and incubated for 1 hour at 4° C. After washing, PE-labeled anti-mouse IgG was added and incubated with cells at 4° C. for 1 hour. Cells were washed twice and MFI of the cells was measured by a flow cytometer and analyzed by FlowJo.

W3256 shows comparable competition ability to the parental antibodies and blocks mouse PD-1 (FIG. 17) binding to PD-L1 with IC₅₀ of ˜1.687 nM.

### 3.8 HUVEC Cell Proliferation Assay

The biological activity of W3256 in VEGF-induced HUVEC proliferation was assessed. HUVEC cells were routinely cultured in ECM+5% FBS+1% ECGS. Sub-confluent cells were harvested by trypsin, diluted to 1×10⁵ cells/mL with ECM+1% FBS+0.05% ECGS. Cells were plated in 96-well clear bottom black plates (Greiner, 655090) at a density of 5000 cells/well. Serial diluted antibodies were added, together with 50 ng/mL of human VEGF (WBP325-hPro1, Sino Biological, 11066-HNAB). The plates were returned to the incubator for 4 days before assessing cell viability using CellTiter Glo (Promega, G7573). Wells with no ligand addition served as control for ligand stimulated cell growth. The effect of the tested antibody on inhibiting ligand stimulated cell growth was calculated by comparing the luminescence values with or without antibody addition (ligand only) after subtracting the background (no ligand) luminescence. Four-parameter non-linear regression analysis was used to obtain proliferation inhibition IC₅₀ values using GraphPad Prism 5 software.

W3256 effectively blocked VEGF induced HUVEC proliferation in a concentration-dependent manner with IC₅₀ of ˜12 nM and maximum inhibition rate of 101% (FIG. 18).

### 3.9 Reporter Gene Assay

Routinely cultured CHOK1-OKT3-PD-L1 cells were harvested by trypsin, and were plated in 96-well clear bottom black plates (Greiner, 655090) at a density of 20000 cells/well. Serial diluted antibodies were added, together with report signal NFAT-RE-Luc2p integrated and full length PD-1-expressing Jurkat cells. The plates were returned to the incubator for 4 hours before 50 μl One-glo luciferase assay buffer/substrate mixture added. The luminescence was measured in a multiwell plate reader M5e.

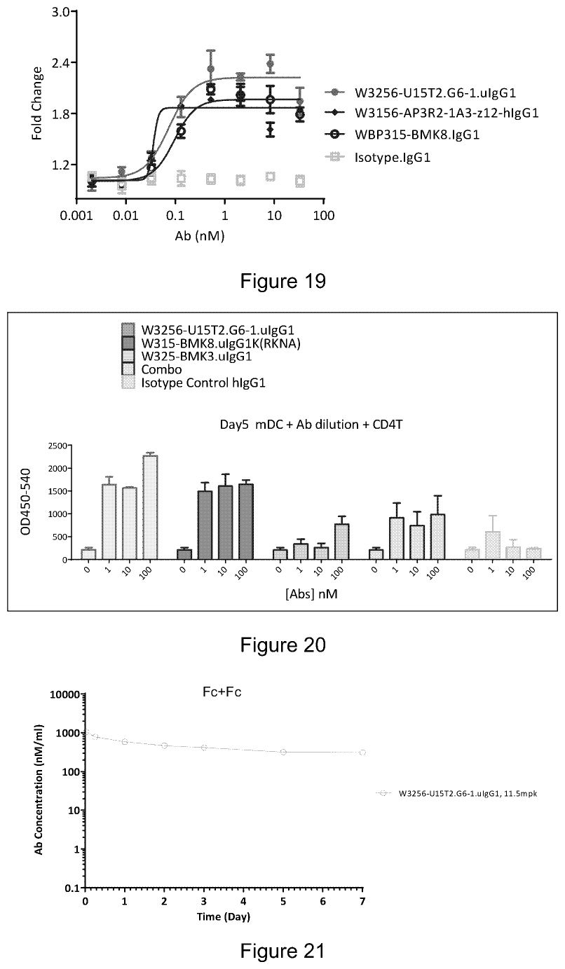

W3256 shows functionality in reporter gene assay (FIG. 19), indicating that the antibody induced PD-1 signaling pathway.

### 3.10 Mixed Lymphocyte Reaction (MLR) Assays

MLR was used to test the agonistic effect of PD-L1 antibodies on cytokine, human IFN-γ secretion and proliferation of activated human CD4⁺ T cells.

Human peripheral blood mononuclear cells (PBMCs) were freshly isolated from healthy donors using Ficoll-Paque PLUS gradient centrifugation. Isolated PBMCs were cultured in complete RPMI-1640 (containing 10% FBS and 1% PS) supplemented with 100 U recombinant human IL-2 (SL PHARM). Monocytes were isolated using Human Monocyte Enrichment Kit according to the manufacturer's instructions. Cell concentration was adjusted to 2×10⁶ cells/ml in complete RPMI-1640 medium supplemented with recombinant human GM-CSF at 800 U/ml and IL-4 at 50 μg/ml. Cells were cultured for 5 to 7 days to differentiate into dendritic cells (DC). Cytokines were replenished every 2-3 days by replacing half of the media with fresh media supplemented with cytokines. 18 to 24 hours before MLR, 1 μg/ml LPS was added to the culture to induce the maturation of the DCs. Human CD4⁺ T cells were isolated using Human CD4⁺ T Cell Enrichment kit according to the manufacturer's protocol.

MLR was set up in 96-well round bottom plates (Nunc, 163320) using complete RPMI-1640 medium. CD4⁺ T cells, various concentrations of antibodies, and mature DCs were added to the plates. The plates were incubated at 37° C., 5% CO₂. IFN-γ production was determined at day 5.

Human IFN-γ was measured by enzyme-linked immunosorbent assay (ELISA) using matched antibody pairs. Recombinant human IFN-γ (PeproTech, 300-02) was used as standards. The plates were pre-coated with capture antibody specific for human IFN-γ (Pierce, M700A). After blocking, standards or samples were pipetted into each well and incubated for 2 hours at ambient temperature. Following removal of the unbound substances, the biotin-conjugated detecting antibody specific for IFN-γ (Pierce, M701B) was added to the wells and incubated for one hour, respectively. The streptavidin conjugated Horseradish Peroxidase (HRP) (Invitrogen, SNN1004) was then added to the wells for 30 minutes incubation at ambient temperature. The color was developed by dispensing TMB substrate, and then stopped by 2M HCl. The absorbance was read at 450 and 540 nm using a Microplate spectrophotometer.

FIG. 20 shows the effect of antibodies on hCD4+ T cell IFN-γ secretion in mixed lymphocyte reaction assay. The result indicated that W3256 could induce IFN-γ secretion in a concentration-dependent manner in MLR.

### Example 4

### In Vivo Antitumor Efficacy Study

### 4.1 Pharmacokinetics Study in Mouse

Male C57BL/6 mice (age of 6-8 weeks, 18-22 g) were purchased from Shanghai SLAC or BK Laboratory Co., LTD., and housed under specific pathogen-free conditions with free access to food and water in the animal facility of WuXi Biologics (Shanghai, China).

Blood samples of Male C57BL/6 mice (n=5) were collected at 0, 0.5, 6, 24, 48, 72, 120, 168 hour after a single i.v. injection of equal molar concentration of W3256 antibody. The concentration of serum antibody was measured by using the human IgG ELISA quantification method and dual-antigen binding ELISA method.

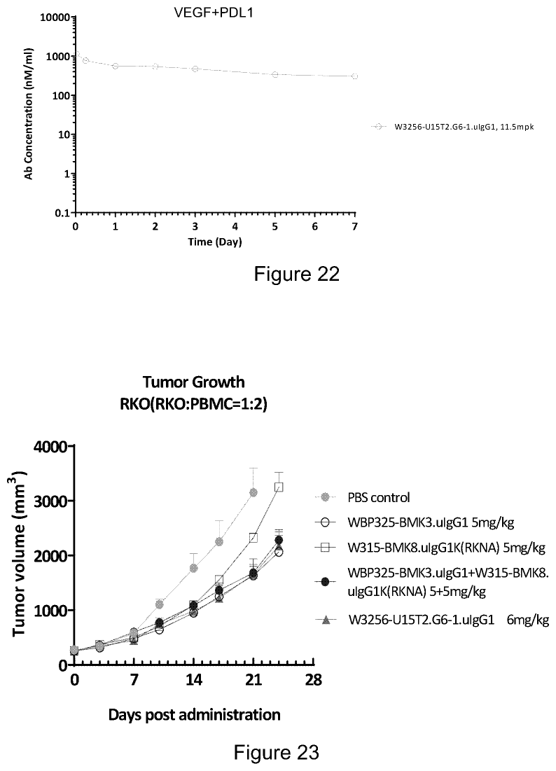

The half-life of W3256 is 129 hours by i.v. injection route at a dose of 11.5 mg/kg according to total IgG binding results (FIG. 21), and 104 hours by i.v. injection route at a dose of 11.5 mg/kg in dual-antigen binding PK assay (FIG. 22).

### 4.2 Efficacy in PBMC-RKO Mouse Model

The RKO tumor cells were maintained in vitro as a monolayer culture in culture medium supplemented with 10% fetal bovine serum, 100 U/ml penicillin and 100 μg/ml streptomycin at 37° C. in an atmosphere of 5% CO₂ in air. The tumor cells were routinely sub-cultured twice weekly by trypsin-EDTA treatment. The cells growing in an exponential growth phase were harvested and counted for tumor inoculation.

Female NCG mice (6-10 weeks old) were purchased from Shanghai SLAC or BK Laboratory Co., LTD., and housed under specific pathogen-free conditions with free access to food and water in the animal facility of WuXi Biologics (Shanghai, China).

Each mouse was inoculated subcutaneously with RKO tumor cells (2×10⁶) in 0.2 ml of PBS with 50% matric gel for tumor development and also i.p. injection of 4×10⁶ PBMC (Hemacare) at Day 0. The treatments were started at Day 7.

The major endpoint was to see if the tumor growth could be delayed or mice could be cured. Tumor size was measured twice weekly using a caliper, and the volume was expressed in mm³ using the formula: V=0.5 a×b² where a and b are the long and short diameters of the tumor, respectively.

Summary statistics, including mean and the standard error of the mean (SEM), are provided for the tumor volume of each group at each time point. Statistical analysis of difference in tumor volume among the groups and the analysis of drug interaction were conducted on the data obtained at the best therapeutic time point after the final dose (the 28th day after start dosing). Two-way ANOVA was performed to compare tumor volume among groups, followed by post-hoc multiple comparison of Dunnett't test (all compared to IgG group). All data were analyzed using SPSS 17.0 or Prism 5. p<0.05 was considered to be statistically significant.

W3256 shows the antitumor efficacy in PBMC-RKO colorectal cancer model in NCG mice, which is significantly better than W315-BMK8 and comparable with W325-BMK3 (FIG. 23).

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure

1. A bispecific antibody or antigen-binding portion thereof, comprising a PD-L1 antigen-binding moiety associated with a VEGF antigen-binding moiety, wherein:
the PD-L1 antigen-binding moiety comprises:
a complementarity determining region (CDR) 1 comprising SEQ ID NO: 1, a CDR2 comprising SEQ ID NO: 2, and a CDR3 comprising SEQ ID NO: 3; and
the VEGF antigen-binding moiety comprises:
a heavy chain complementarity determining region (HCDR) 1 comprising SEQ ID NO: 4, a HCDR2 comprising SEQ ID NO: 5, a HCDR3 comprising SEQ ID NO: 6, a light chain complementarity determining region (LCDR) 1 comprising SEQ ID NO: 7, a LCDR2 comprising SEQ ID NO: 8, and a LCDR3 comprising SEQ ID NO: 9.

2. The bispecific antibody or antigen-binding portion thereof of claim 1, wherein the PD-L1 antigen-binding moiety comprises:
a variable domain comprising the amino acid sequence of SEQ ID NO: 10 or an amino acid sequence at least 85%, 90%, or 95% identical to SEQ ID NO: 10.

3. The bispecific antibody or antigen-binding portion thereof of claim 1, wherein the VEGF antigen-binding moiety comprises:
a heavy chain variable domain comprising the amino acid sequence of SEQ ID NO: 11 or an amino acid sequence at least 85%, 90%, or 95% identical to SEQ ID NO: 11; and
a light chain variable domain comprising the amino acid sequence of SEQ ID NO:12 or an amino acid sequence at least 85%, 90%, or 95% identical to SEQ ID NO: 12.

4. (canceled)

5. The bispecific antibody or antigen-binding portion thereof of claim 1, wherein the PD-L1 antigen-binding moiety is from a VHH antibody derived from a camelid animal, comprising an alpaca or a llama.

6. (canceled)

7. The bispecific antibody or antigen-binding portion thereof of claim 1, wherein the PD-L1 antigen-binding moiety is operably linked to the N terminal of the light chain or heavy chain of the VEGF antigen-binding moiety, optionally via a linker.

8. The bispecific antibody or antigen-binding portion thereof of claim 5, wherein the linker comprises or consists of 1 to 4 copies of GGGGS (G4S).

9. The bispecific antibody or antigen-binding portion thereof of claim 1, comprising a heavy chain and a light chain, wherein:
the heavy chain comprises domains operably linked as in VH-CH1-hinge-Fc, wherein the VH-CH1 is from the VEGF antigen binding moiety; and
the light chain comprises domains operably linked as in VHH-VL-CL, wherein the VHH is from the PD-L1 antigen binding moiety and the VL-CL is from the VEGF antigen binding moiety.

10. The bispecific antibody or antigen-binding portion thereof of claim 7, wherein the Fc region is a human IgG Fc region, preferably a human IgG1 Fc region.

11. The bispecific antibody or antigen-binding portion thereof of claim 7, wherein the heavy chain comprises SEQ ID NO: 13, and the light chain comprises SEQ ID NO: 14.

12. (canceled)

13. An isolated nucleic acid molecule, comprising a nucleic acid sequence encoding the bispecific antibody or the antigen-binding portion thereof of claim 7.

14. A vector comprising the nucleic acid molecule of claim 10.

15. A host cell comprising the nucleic acid molecule of claim 10.

16. A pharmaceutical composition comprising the bispecific antibody or the antigen-binding portion thereof of claim 1 and a pharmaceutically acceptable carrier.

17. A method for producing the bispecific antibody or the antigen-binding portion thereof of claim 1, comprising the steps of:
culturing a host cell comprising a nucleic acid molecule(s) encoding the bispecific antibody or the antigen-binding portion thereof; and
isolating the antibody or antigen-binding portion thereof from the host cell.

18. A method for modulating an immune response in a subject, comprising administering to the subject the bispecific antibody or the antigen-binding portion thereof of claim 1 to the subject.

19. (canceled)

20. A method for preventing or treating cancer in a subject, comprising administering an effective amount of the bispecific antibody or the antigen-binding portion thereof of claim 1 to the subject.

21. The method of claim 16, wherein the cancer is selected from colon cancer, colorectal cancer, breast cancer, lung cancer, cervical cancer, renal cancer, glioblastoma, ovarian cancer, pancreatic cancer, prostate cancer, esophageal cancer, gastric cancer, lymphoma, melanoma, liver cancer, and head and neck cancer.

22. The method of claim 17, wherein the cancer is colon cancer or colorectal cancer.

23. The method of claim 16, wherein the bispecific antibody or antigen-binding portion thereof is administered in combination with a chemotherapeutic agent, radiation and/or other agents for use in cancer immunotherapy.

24-25. (canceled)

26. A kit comprising the bispecific antibody or antigen-binding portion thereof of claim 1.
